(19) United States
(12) Patent Application Publication
(10) Pub. No.: US 2025/0049476 A1
(43) Pub. Date: Feb. 13, 2025

(54) Image-Based Implant Length Determination and Associated Systems, Devices, and Methods

(71) Applicant: ORTHOFIX US LLC, Lewisville, TX (US)

(72) Inventors: John LOVELL, North Bergen, NJ (US); John LYON, Lewisville, TX (US); James Sterling DENTON, Lewisville, TX (US); Jose Luis CHARVET, Lewisville, TX (US); Lena LOUTFY, Lewisville, TX (US); Parul JAIN, Lewisville, TX (US)

(21) Appl. No.: 18/718,312

(22) PCT Filed: Nov. 22, 2022
(86) PCT No.: PCT/US2022/050687

§ 371 Date: Jun. 10, 2024

Related U.S. Application Data
(60) Provisional application No. 63/292,649, filed on Dec. 22, 2021.

(51) Int. Cl. A61B 17/70; A61B 34/10; A61B 90/00

(52) U.S. Cl. CPC A61B 17/7014; A61B 34/10; A61B 90/37; A61B 2034/108; A61B 2090/376

(57) ABSTRACT

Systems, methods, and devices for determining a length of a connecting rod are described. In some aspects, a method includes obtaining at least one radiographic image representative of a first medical device inserted into a body of a patient, and determining a calibration factor of the at least one radiographic image. The method may further include receiving one or more rod length selection parameters, and determining, based on the at least one radiographic image, a first rod connection point and a second rod connection point. The method may further include determining, based on the first rod connection point, the second rod connection point, and the one or more rod selection parameters, a length of the connecting rod. The method may further include outputting the length of the rod to a display.

### TECHNICAL FIELD

The present disclosure relates generally to systems, devices, and methods for determining or computing a length of an implant using image processing and analysis.

### BACKGROUND

The bones and connective tissue of an adult human spinal column consists of more than 20 discrete bones coupled sequentially to one another by a tri-joint complex. The complex consists of an anterior disc and two posterior facet joints. The anterior discs of adjacent bones are cushioned by cartilage spacers referred to as intervertebral discs. The over 20 bones of the spinal column are anatomically categorized as one of four classifications: cervical, thoracic, lumbar, or sacral. The cervical portion of the spine which comprises the top of the spine up to the base of the skull, includes the first 7 vertebrae. The intermediate 12 bones are thoracic vertebrae, and connect to the lower spine comprising the 5 lumbar vertebrae. The base of the spine are sacral bones, including the coccyx.

The spinal column of bones is highly complex in that it includes over 20 bones coupled to one another, housing and protecting critical elements of the nervous system having innumerable peripheral nerves and circulatory bodies in close proximity. Despite its complexity, the spine is a highly flexible structure, capable of a high degree of curvature and twist in nearly every direction.

Genetic or developmental irregularities, trauma, chronic stress, tumors and disease, however, can result in spinal pathologies which either limit this range of motion or threaten the critical elements of the nervous system housed within the spinal column. A variety of systems have been disclosed in the art which achieve immobilization by implanting artificial assemblies in or on the spinal column. These assemblies may be classified as anterior, posterior or lateral implants. Lateral and anterior assemblies are coupled to the anterior portion of the spine which is in the sequence of vertebral bodies. Posterior implants generally comprise pairs of rods (“bilateral spinal support rods”), which are aligned along the axis which the bones are to be disposed, and which are then attached to the spinal column by screws which are inserted through pedicles.

Spinal stabilization or immobilization assemblies involve patient-specific geometries. Further, the size of some of the components, such as the bilateral spinal support rods, may not be determinable until the screws are positioned in the pedicles and the distance between the screws is known. Other variables that affect the size and/or length of an implant include the patient's lordosis or spinal curvature, the tools and instruments used in the procedure, and the doctor's preferences, such as the overhang of the spinal support rods on either end of the assembly.

### SUMMARY

The present disclosure describes systems, devices, and methods for image-based determination of implant lengths and/or other geometries. In one embodiment, a mobile computing device, such as a smartphone or a tablet, receives a radiographic image of a patient's spine, including at least one pedicle screw assembly. The mobile computing device may calibrate the image dimensions based on a calibration feature in the image of a known-dimension. For example, the calibration feature may include a radiographic (e.g., radiopaque) marker positioned on the patient, the radiographic marker associated with a known width, height, diameter, and/or other dimension. The mobile computing device may further receive one or more user inputs indicating rod length selection parameters. The rod length selection parameters may include an optional overhang measurement, screw length, estimated lordosis, and/or any other suitable parameter. The mobile computing device identifies outer rod connection points in at least one of the radiographic images, the outer rod connection points associated with the desired span of the rod in the patient's spine. Based on the calibrated image dimensions, the outer rod connection points, and the rod length selection parameters, the mobile computing device determines or selects a rod length, and outputs the rod length to a display. In some aspects, the mobile computing device may include a memory device storing a database of available rod lengths and/or rod configurations, and select the rod length from the database.

In some aspects, the mobile communication device may be configured to output a graphical representation of the selected rod to the display, where the graphical representation is overlaid on at least one of the radiographic images to scale with the spine and pedicle screws. The user may then manipulate the graphical representation of the rod by, for example, translating and/or rotating the graphical representation of the rod into place at the desired connection points. If the curvature of the graphical representation of the rod does not match the curvature of the spine in the image, the user can manipulate the shape of the graphical representation to adjust the rod curvature. For example, the user may use a touch screen interface to adjust the curvature of the graphical representation of the rod. In some instances, the mobile computing device may update or reselect the rod length based on the rod curvature adjustment prompted by the user inputs.

According to an embodiment of the present disclosure, a method of determining a size of a connecting rod includes: obtaining, by an image acquisition unit of a mobile computing device, at least one radiographic image, wherein the at least one radiographic image is representative of a first medical device inserted into a body of a patient and a calibration marker; determining, by a processor of the mobile computing device based on the calibration marker in the at least one radiographic image, a calibration factor of the at least one radiographic image; receiving, from a user interface of the mobile computing device, one or more rod length selection parameters; determining, based on a shape of the first medical device, a first rod connection point associated with the first medical device; determining, based on at least one of a shape of a second medical device in the radiographic image or a first rod selection parameter of the one or more rod selection parameters, a second rod connection point; determining, by the processor of the mobile computing device based on the first rod connection point, the second rod connection point, the calibration factor of the at least one radiographic image, and the one or more rod selection parameters, a length of the connecting rod associated with a distance between the first rod connection point and the second rod connection point; and outputting, to a display, a visual representation of the length of the rod.

In some aspects, the at least one radiographic image is representative of the first medical device and the second medical device implanted into the patient, the first medical device includes a first radiographic indicia and the second medical device includes a second radiographic indicia, the method further includes: determining, by the processor of the mobile computing device, a deformation of the first indicia; and determining by the processor of the mobile computing device, a deformation of the second indicia; the determining the first rod connection point is based on the first deformation; and the determining the second rod connection point is further based on the second deformation. In some aspects, the method further includes: determining a first relative length of the first indicia; and determining a second relative length of the second indicia, wherein the determining the first rod connection point is based on the first relative length of the first indicia, and wherein the determining the second rod connection point is based on the second relative length of the second indicia. In some aspects, the first indicia comprises a first plurality of indicia features, the determining the first deformation of the first indicia comprises determining: a first relative distance between a first indicia feature and a second indicia feature of the first plurality of indicia features; and a second relative distance between a third indicia feature and a fourth indicia feature of the first plurality of indicia features. In some aspects, the determining the first deformation of the first indicia comprises: determining a first dimension of the first indicia feature; determining a second dimension of the second indicia feature; and comparing the first dimension to the second dimension. In some aspects, the receiving the one or more rod selection parameters comprises receiving, from the user interface of the mobile computing device, a first input indicating the second rod connection point in the one or more radiographic images. In some aspects, the receiving the one or more rod selection parameters comprises receiving, from the user interface of the mobile computing device, a second input indicating an overhang length of the connecting rod. In some aspects, the determining the rod length is based on the second input.

In some aspects, the receiving the one or more rod selection parameters comprises receiving, from the user interface of the mobile computing device, a third input indicating at least one of: a type of the first medical device; or a dimension of a fixation device associated with the implant, wherein the determining the rod length is based on the third input. In some aspects, the determining the rod length comprises selecting, based on the first rod connection point and the second rod connection point, a stored rod length from a database including a plurality of stored rod lengths. In some aspects, the method further includes: determining, by an orientation sensor of the mobile computing device, an orientation of the mobile computing device, wherein the determining the calibration factor is based on the orientation of the mobile computing device. In some aspects, the method further includes: outputting, to the user interface of the mobile computing device, a graphical indicator of a rod overlayed on a radiographic image of the one or more radiographic images, wherein the graphical indicator is scaled relative to the radiographic image based on the calibration factor. In some aspects, the method further includes: receiving, from the user interface of the mobile computing device, a fourth input indicating an adjustment to a curvature of the connecting rod; and updating the graphical indicator based on the adjustment to the curvature of the connecting rod. In some aspects, the method further includes: determining, based on the fourth input, an updated rod length; and outputting, to the user interface, a visual indication of the updated rod length.

According to another embodiment of the present disclosure, a mobile computing device includes: an image acquisition unit configured to obtain at least one radiographic image, wherein the at least one radiographic image is representative of a first medical device inserted into a body of a patient and a calibration marker; a user interface; and a processor in communication with the image acquisition unit and the user interface, the processor configured to: determine, based on the calibration marker in the at least one radiographic image, a calibration factor of the at least one radiographic image; receive, from a user interface of the mobile computing device, one or more rod length selection parameters; determine, based on a shape of the first medical device, a rod connection point associated with the first medical device; determine, based on at least one of a shape of a second medical device in the radiographic image or a first rod selection parameter of the one or more rod selection parameters, a second rod connection point; determine, based on the first rod connection point, the second rod connection point, the calibration factor of the at least one radiographic image, and the one or more rod selection parameters, a length of the connecting rod associated with a distance between the first rod connection point and the second rod connection point; and output, to the user interface, a visual representation of the length of the rod.

In some aspects, the at least one radiographic image is representative of the first medical device and the second medical device implanted into the patient, the first medical device includes a first radiographic indicia and the second medical device includes a second radiographic indicia. In some aspects, the processor is further configured to: determine, by the processor of the mobile computing device, a deformation of the first indicia; and determine by the processor of the mobile computing device, a deformation of the second indicia; the processor is configured to determine the first rod connection point is based on the first deformation. In some aspects, the processor is configured to determine the second rod connection point is further based on the second deformation. In some aspects, the processor is configured to: determine a first relative length of the first indicia; and determine a second relative length of the second indicia, wherein the processor is configured to determine the first rod connection point is based on the first relative length of the first indicia. In some aspects, the processor is configured to determine the second rod connection point is based on the second relative length of the second indicia. In some aspects, the first indicia comprises a first plurality of indicia features, and the processor is configured to determine the first deformation of the first indicia based on: a first relative distance between a first indicia feature and a second indicia feature of the first plurality of indicia features; and a second relative distance between a third indicia feature and a fourth indicia feature of the first plurality of indicia features. In some aspects, the processor is configured to determine the first deformation of the first indicia based on: a first dimension of the first indicia feature; a second dimension of the second indicia feature; and a comparison of the first dimension to the second dimension.

In some aspects, the processor configured to receive the one or more rod selection parameters comprises the processor configured to receive, from the user interface of the mobile computing device, a first input indicating the second rod connection point in the one or more radiographic images. In some aspects, the processor configured to receive the one or more rod selection parameters comprises the processor configured to receive, from the user interface of the mobile computing device, a second input indicating an overhang length of the connecting rod. In some aspects, the processor is configured to determine the rod length based on the second input. In some aspects, the processor configured to receive the one or more rod selection parameters comprises the processor configured to receive, from the user interface of the mobile computing device, a third input indicating at least one of: a type of the implant; or a dimension of a fixation device associated with the implant. In some aspects, the processor is configured to determine the rod length based on the third input. In some aspects, the processor configured to determine the rod length comprises the processor configured to select, based on the first rod connection point and the second rod connection point, a stored rod length from a database including a plurality of stored rod lengths. In some aspects, the processor is further configured to: determine, based on orientation data from an orientation sensor of the mobile computing device, an orientation of the mobile computing device. In some aspects, the processor is configured to determine the calibration factor further based on the orientation of the mobile computing device. In some aspects, the processor is further configured to: output, to the user interface of the mobile computing device, a graphical indicator of a rod overlayed on a radiographic image of the one or more radiographic images, wherein the graphical indicator is scaled relative to the radiographic image based on the calibration factor. In some aspects, the processor is further configured to: receive, from the user interface of the mobile computing device, a fourth input indicating an adjustment to a curvature of the connecting rod; and update the graphical indicator based on the adjustment to the curvature of the connecting rod. In some aspects, the processor is further configured to: determine, based on the fourth input, an updated rod length; and output, to the user interface, a visual indication of the updated rod length.

Additional aspects, features, and advantages of the present disclosure will become apparent from the following detailed description.

### BRIEF DESCRIPTION OF THE DRAWINGS

For a more complete understanding of the present disclosure and its advantages, reference is now made to the following description taken in conjunction with the accompanying drawings, in which like reference numbers indicate like features, and:

FIG. 1 is a diagrammatic view of a system for determining a length of a spinal stabilization rod according to aspects of the present disclosure.

FIG. 2 is a lateral view of a radiographic image of a spinal stabilization system connected to a patient's spine according to aspects of the present disclosure.

FIG. 3 is an anteroposterior (A/P) view of a radiographic image of a spinal stabilization system connected to a patient's spine according to aspects of the present disclosure.

FIG. 4 is a lateral view of a radiographic image of a spinal stabilization system connected to a patient's spine according to aspects of the present disclosure.

FIG. 5A is a diagrammatic view of a calibration marking pattern according to aspects of the present disclosure.

FIG. 5B is a diagrammatic view of a calibration marking pattern according to aspects of the present disclosure.

FIG. 5C is a diagrammatic view of a calibration marking pattern according to aspects of the present disclosure.

FIG. 5D is a diagrammatic view of a calibration marking pattern according to aspects of the present disclosure.

FIG. 6 is a flow diagram of a method for determining a length of a spinal stabilization rod according to aspects of the present disclosure.

FIGS. 7A-7L illustrate various mobile computing device user interfaces associated with the method of FIG. 6 according to aspects of the present disclosure.

FIG. 8 is a flow diagram of a method for determining a length of a spinal stabilization rod according to aspects of the present disclosure.

FIG. 9 is a schematic diagram of a mobile computing device according to aspects of the present disclosure.

FIG. 10 is a flow diagram of a method for removing patient-identifying information (PII) from medical image data according to aspects of the present disclosure.

FIGS. 11A and 11B illustrate mobile computing device user interfaces associated with the method of FIG. 10 according to aspects of the present disclosure.

FIG. 12 illustrates an image of an anatomy before and after a PII data removal process according to aspects of the present disclosure.

Although similar reference numbers may be used to refer to similar elements for convenience, it can be appreciated that each of the various example embodiments may be considered to be distinct variations.

### DETAILED DESCRIPTION

Exemplary embodiments will now be described hereinafter with reference to the accompanying figures, which form a part hereof, and which illustrate examples by which the exemplary embodiments, and equivalents thereof, may be practiced. As used in the disclosures and the appended claims, the terms “embodiment,” “example embodiment” and “exemplary embodiment” do not necessarily refer to a single embodiment, although it may, and various example embodiments, and equivalents thereof, may be readily combined and interchanged, without departing from the scope or spirit of present embodiments. Furthermore, the terminology as used herein is for the purpose of describing example embodiments only and is not intended to be limitations of the embodiments. In this respect, as used herein, the term “plate” may refer to any substantially flat structure or any other three-dimensional structure, and equivalents thereof, including those structures having one or more portions that are not substantially flat along one or more axis. Furthermore, as used herein, the terms “opening,” “recess,” “aperture,” and equivalents thereof, may include any hole, space, area, indentation, channel, slot, bore, and equivalents thereof, that is substantially round, oval, square, rectangular, hexagonal, and/or of any other shape, and/or combinations thereof, and may be defined by a partial, substantial or complete surrounding of a material surface. Furthermore, as used herein, the term “in” may include “in” and “on,” and the terms “a,” “an” and “the” may include singular and plural references. Furthermore, as used herein, the term “by” may also mean “from,” depending on the context. Furthermore, as used herein, the term “if” may also mean “when” or “upon,” depending on the context. Furthermore, as used herein, the words “and/or” may refer to and encompass any and all possible combinations of one or more of the associated listed items.

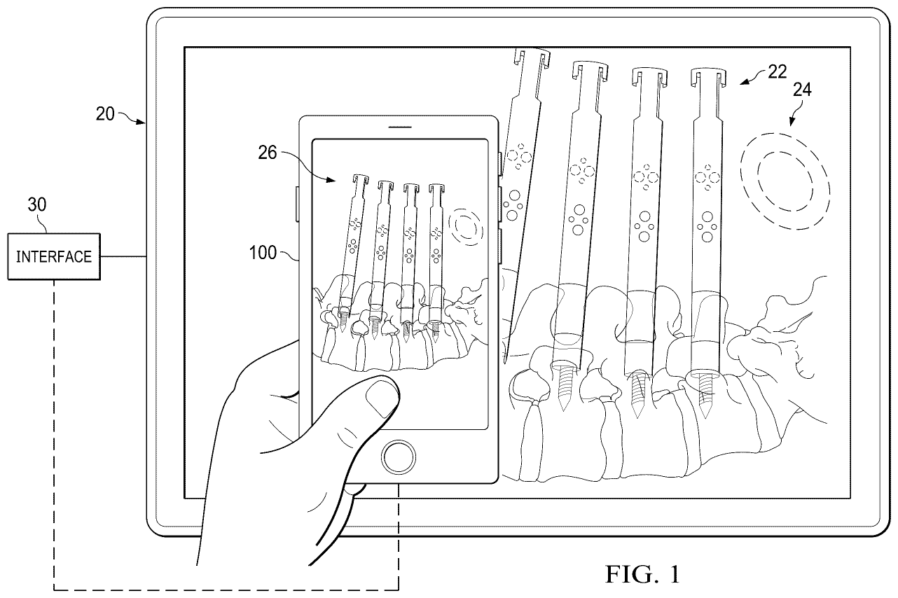

FIG. 1 is a diagrammatic view of a system for image-based implant length selection, according to an embodiment of the present disclosure. The system includes a mobile computing device 100, a display 20, and an interface 30. In the illustrated embodiment, the mobile computing device 100 comprises a smartphone. The smartphone includes a touch screen display, a camera, a processor, and a memory configured to store images and instructions or computer programs executable by the processor. The mobile computer device 100 may include its own data interface for receiving data such as image data, measurement data, tooling parameters, implant size databases, and/or any other suitable type of data. The display 20 may be in communication with a radiography device, such as an x-ray imaging device. In some aspects, the display 20 may be configured to receive and display medical images, such as x-ray images, fluoroscopic images, angiographic images, computed tomography (CT) images, magnetic resonance imaging (MRI) images, and/or any other suitable type of medical image.

Referring to FIG. 1, the display 20 is displaying an image that includes a reduction screw assembly 22 and a calibration marker 24. In some aspects, the image on the display 20 includes a fluoroscopic image of the patient's spine and a plurality of pedicle reduction screw assemblies 22. The reduction screw assemblies 22 include bone screws configured to be driven into the patient's vertebral bodies, and receiver assemblies or towers extending proximally from the screws. As explained further below, once the surgeon has placed the reduction screw assemblies into the vertebral bodies, the surgeon may guide a rigid connecting rod through vertical slots or channels in each of the receiver assemblies. The connecting rod can then be lowered into a saddle or base of the receiver assemblies, and fixed into place with a set screw or compression screw. The connecting rod may come pre-configured with a curve to account for the lordosis of the patient's spine. Further, the connecting rod may be included in an assembly or kit including a variety of connecting rods having different lengths, thicknesses, and/or curvatures.

In some aspects, the length of the connecting rod may be based on a variety of factors, including anatomical factors and implant dimensions. For example, the length of the connecting rod may correspond to the distance between the lowest-placed pedicle screw assembly and the highest-placed pedicle screw assembly. In this regard, the surgeon may not select the rod length until at least some of the pedicle screw assemblies have been placed. Thus, the connecting rod may be selected during surgery, which can further complicate the procedure. The present disclosure provides systems, devices, and methods for determining the length of a connecting rod based on image data representative of a patient's spine, and at least one implant assembly (e.g., pedicle screw assembly), connected to the spine. In this regard, the mobile computing device 100 can be configured to receive the image of the screw assemblies 22 and calibration marker 24 on the display 20, and to process the image to identify the outer connection points for calculating the length of the connecting rod. The mobile computing device 100 may output a user interface 26 including a reproduction or duplication of the image on the display 20. The user interface 26 may assist the user to control the mobile computing device 100 during one or more steps of a rod length calculation method. The calculation may be based on determining a relative size (e.g., in pixels) and/or deformation of the calibration marker 24, and comparing the determined size to a known or stored size of the calibration marker. In some embodiments, the mobile computing device 100 may obtain the image from the display 20 using a camera of the mobile computing device 100. In other embodiments, the mobile computing device 100 may receive image data as obtained by the medical imaging device via the interface 30. The interface 30 may provide for a wired and/or a wireless link with the mobile computing device 100.

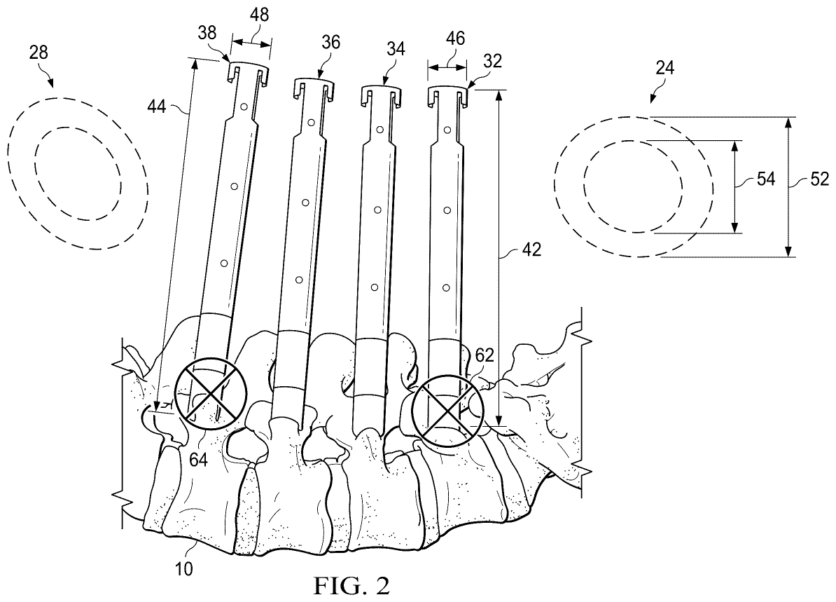

FIG. 2 is an illustration of an x-ray image of a bone fixation system connected to a patient's spine 10. More specifically, FIG. 2 shows a lateral view of the bone fixation system and the spine 10. The bone fixation system includes a plurality of reduction screw tower bodies or assemblies 32, 34, 36, 38 extending proximally of bone screws driven into respective vertebrae of the spine 10. Each of the tower bodies 32, 34, 36, 38 includes two prongs on either side of a channel or slot. The channel or slot may be sized, shaped, and otherwise structurally configured to receive a connecting rod. Each of the tower bodies 32, 34, 36, 38 is associated with a corresponding rod connection point, including the rod connection points 62 and 64. The rod connection points 62 and 64 represent the outermost connection points, as they are associated with the outermost reduction screw tower bodies 32, 38. In some aspects, the rod connection points 62, 64 are disposed at a base of the tower bodies 32, 38. Each of the tower bodies 32, 34, 36, 38 may include a U-shaped slot or saddle configured to seat the connecting rod for fixation to the bodies 32, 34, 36, 38.

The tower bodies 32, 34, 36, 38 each have a corresponding length and width. For example, the lowest tower body 32 has a first length 42 and a first width 46. The highest tower body 28 has a second length 44 and a second width 48. The terms “lowest” and “highest” may refer to the relative placement on the spine, where the higher bodies are closer to the patient's head, and the lower bodies are close to the patient's tailbone.

Two calibration markers 24, 28 are also shown in the image. The markers 24, 28 are shown having circular donut shapes. In other embodiments, one or both of the markers 24, 28 have solid circular shapes, rectangular shapes, triangular shapes, hexagonal shapes, elliptical shapes, and/or any other suitable shape. The calibration makers 24, 28 may be adhesive markers including a radiopaque material and an adhesive backing to attach to the patient's skin. The calibration makers 24, 28, have one or more known dimensions. For example, each calibration marker may have an outside diameter 52 and an inside diameter 54. In other embodiments, each calibration maker may have a height and a width. The image shows the calibration markers 24, 28 as somewhat skewed or deformed. This may be a result of the relative orientations of the calibration markers 24, 28 with respect to the imaging device.

In some embodiments, a computing device, such as the mobile computing device 100 shown in FIG. 1, may be configured to receive an image including the implants and markers shown in FIG. 2, and to determine at least the outer connection points 62, 64 by analyzing the image data. In this regard, the computing device may be configured to calibrate the image, or the dimensions of the image features, based on the calibration markers 24, 28. For example, the computing device may determine a relative size (e.g., width, diameter 52, etc.) of one or more of the markers 24, 28 in pixels, and calculate a conversion ratio for the size of the features (e.g., length 44) in pixels to the size of the features in absolute measurement units, such as millimeters or inches. The computing device may identify or locate the rod connection points based on identifying the outermost tower assemblies 32, 38, locating one or more boundaries of the tower assemblies 32, 38, and retrieving, from memory, known geometrical characteristics of the tower assemblies to identify the relative locations of the rod connection points with respect to one or more reference points of the tower assemblies (e.g., top, bottom, lateral edges, screw tips, tower base, etc.). The computing device may also receive user inputs indicating one or more rod selection parameters. For example, the computing device may receive, from a touch screen interface, keyboard, voice control module, or any other suitable interfaces, indications of one or more of an optional overhang, screw size, additional curvature, and/or any other suitable input.

Based on the located rod connection points 62, 64 derived using image processing, and the rod selection parameters, the computing device may determine or select a rod length for implanting into the patient to span the rod connection points 62, 64 according to the rod selection parameters (e.g., optional overhang, additional curvature). The computing device may then output an indication of the rod length to a user interface device, such as a touchscreen display or an external display.

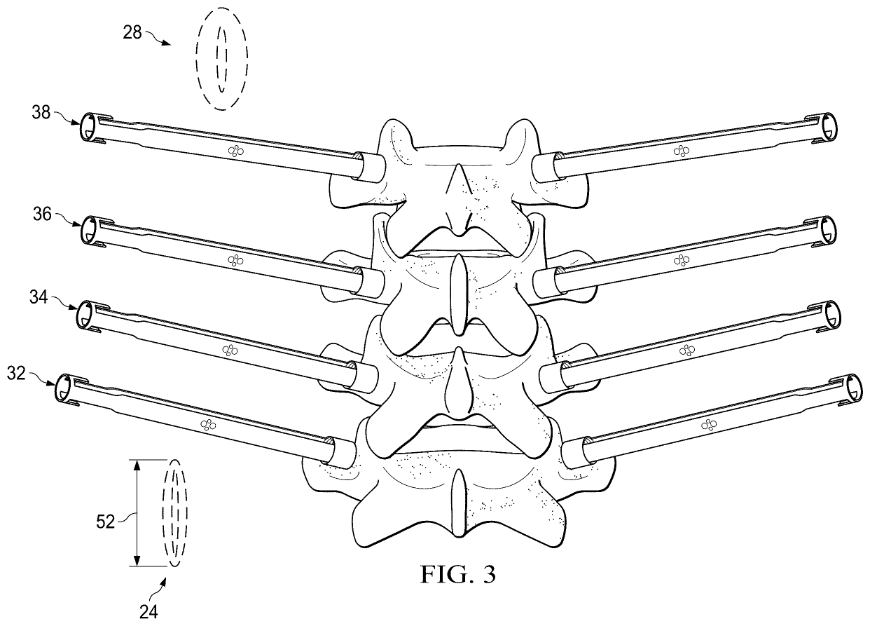

In some aspects, multiple views of the spine 10 and spinal fixation assembly may be used or input into the computing device for determining the rod length. In this regard, FIG. 3 shows an anterior-posterior (AP) fluoroscopic image of the spine 10 and fixation assembly including the receiver tower bodies 32, 34, 36, 38, and the calibration markers 24, 28. In some aspects, the multiple views may be used to better determine rod connection points to account for tiling of the tower bodies 32, 34, 36, 38. In some aspects, the AP view, combined with the lateral view shown in FIG. 2, may be used to better determine the calibration factor based on the shape and dimensions of the calibration markers 24, 28 from each view. In some aspects, a parallax effect derived from the multiple views can be used to improve the accuracy of the rod connection point determination and/or the rod length calculation.

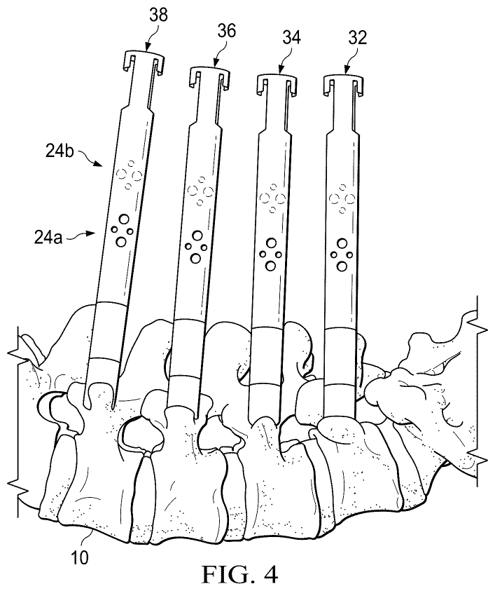

In another embodiment of the present disclosure, radiographic markers can be incorporated into the pedicle screw receiver assemblies, where the radiographic markers are used as calibration markers to determine a conversion or calibration factor for the image. FIG. 4 illustrates a radiographic image of a spinal fixation system attached to a patient's spine 10. Similar to the system shown in FIGS. 2 and 3, the spinal fixation system of FIG. 4 includes a plurality of pedicle screw assemblies, which include bone screws driven into adjacent vertebrae, and receiver tower bodies 32, 34, 36, 38 extending vertically from the heads of the bone screws. Each of the receiver tower bodies 32, 34, 36, 38 includes calibration markings 24 formed by drilling holes according to the pattern shown. The pattern of each set of calibration markings 24a, 24b may indicate the orientation of the receiver tower bodies 32, 34, 36, 38 with respect to the imaging device. Similar to the markers 24, 28 shown above with respect to FIGS. 2 and 3, the markings 24a, 24b may be used to calibrate the image and to convert measurements in pixels to measurements in absolute terms, such as millimeters or inches. Further, the computing device may be configured to determine an amount of tilt for each degree of freedom based on the shapes and sizes of each portion of the markings 24a, 24b.

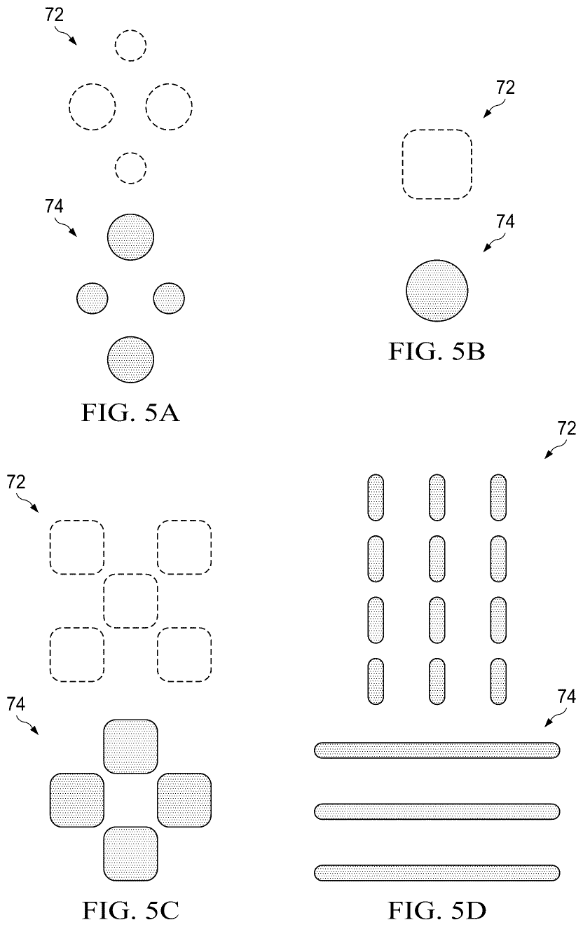

FIGS. 5A-5D illustrate various calibration marking patterns 72, 74 according to various embodiments of the present disclosure. Each of the marking patterns 72, 74 may be incorporated into a spinal implant device, such as a pedicle screw assembly. For example, the calibration marking patterns 72, 74 may be incorporated into a receiver body tower. In some embodiments, the patterns can be incorporated into the implants by drilling or otherwise removing material based on the indicated patterns. In other embodiments, the calibration marking patterns 72, 74 may be incorporated into the implants by affixing radiopaque markings one the components.

The markings 72, 74 shown in FIG. 5A include patterns of larger and smaller circles and/or ellipses arranged in a diamond pattern. The upper markings 72, represented with dotted or dashed lines, may illustrate the markings shown on the opposite side or prong of a tower body. The pattern shown may indicate which side of the device is facing the imaging device. Further, the computing device may determine the tilt of the tower bodies and the location of the connection points based on the deformations of the markings 72, 74, for example. In some aspects, the computing device may be configured to identify a boundary of each portion of the markings 72, 74, and obtain one or more measurements of each portion to determine the deformation. For example, the computing device may be configured to determine a height, width, eccentricity, diameter, and/or any other suitable measurements to determine the tilt of the device and/or the location of the corresponding rod connection point.

FIG. 5B illustrates a pair of calibration markings 72, 74 according to another embodiment of the present disclosure. A first calibration marking 72 includes a rectangular shape, and a second calibration marking 74 includes a circular shape. As above, the markings 72, 74 may be formed by drilling, cutting, or otherwise removing material from the spinal fixation device.

FIG. 5C illustrates a pair of calibration markings 72, 74 according to another embodiment of the present disclosure. A first calibration marking pattern 72 includes a first checkerboard pattern, and a second calibration marking pattern 74 includes a second checkerboard pattern that is the inverse of the first checkerboard pattern. As above, the marking patterns 72, 74 may be formed by drilling, cutting, or otherwise removing material from the spinal fixation device.

FIG. 5D illustrates a pair of calibration markings 72, 74 according to another embodiment of the present disclosure. A first calibration marking pattern 72 includes a series of parallel, vertical lines, and a second calibration marking pattern 74 includes a series of parallel, horizontal lines. As above, the marking patterns 72, 74 may be formed by drilling, cutting, or otherwise removing material from the spinal fixation device.

It will be understood that the patterns of the markings 72, 74 shown in FIGS. 5A-5D are not intended to be limiting. The present disclosure contemplates other patterns and shapes of calibration markings that are different in one or more aspects from the patterns shown above.

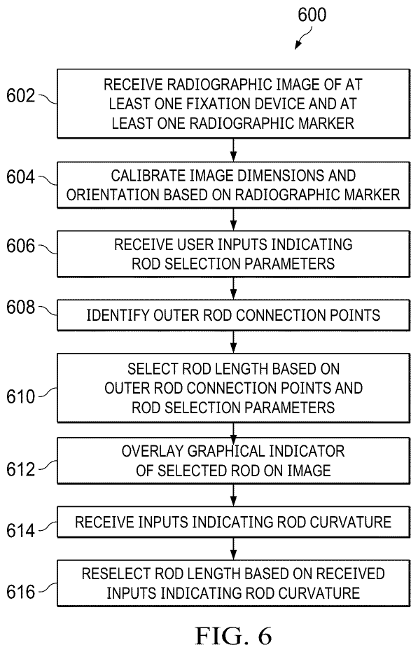

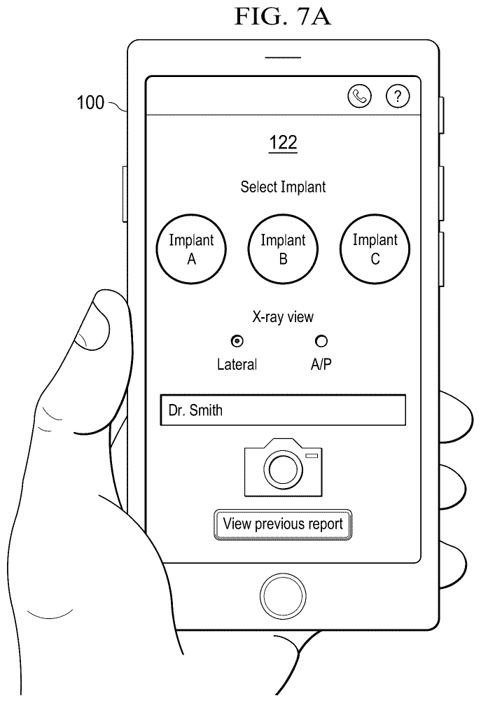

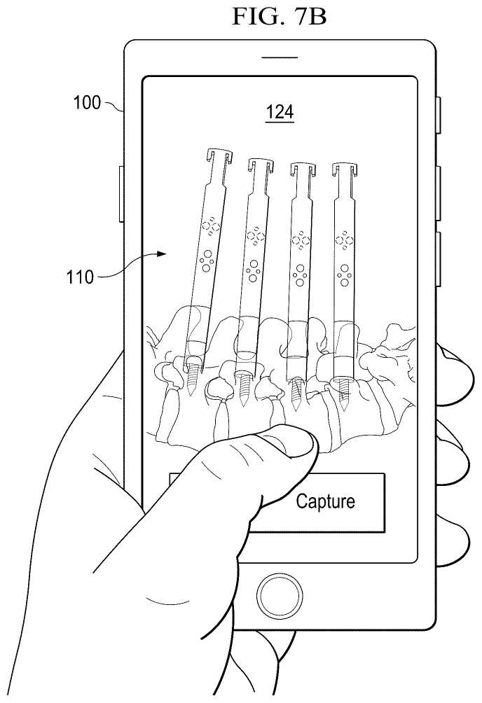

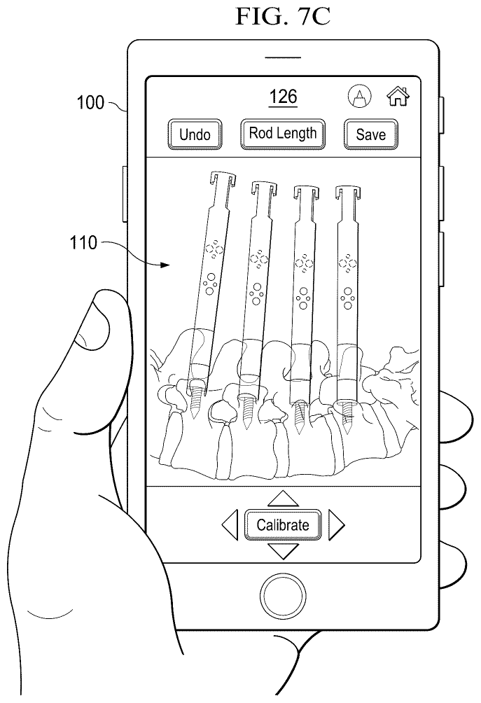

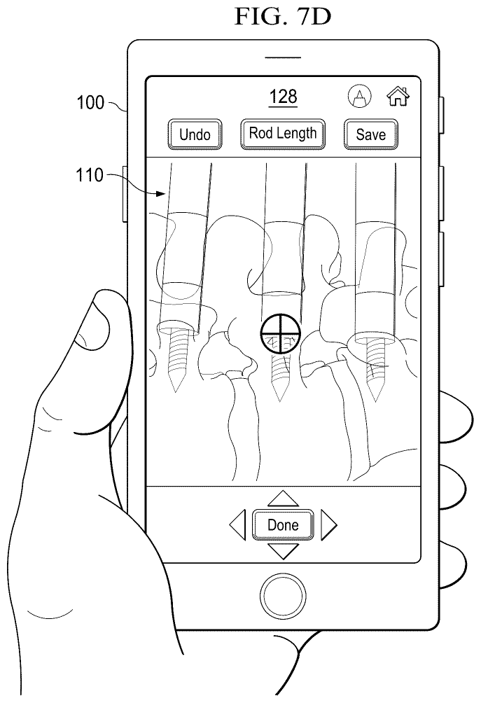

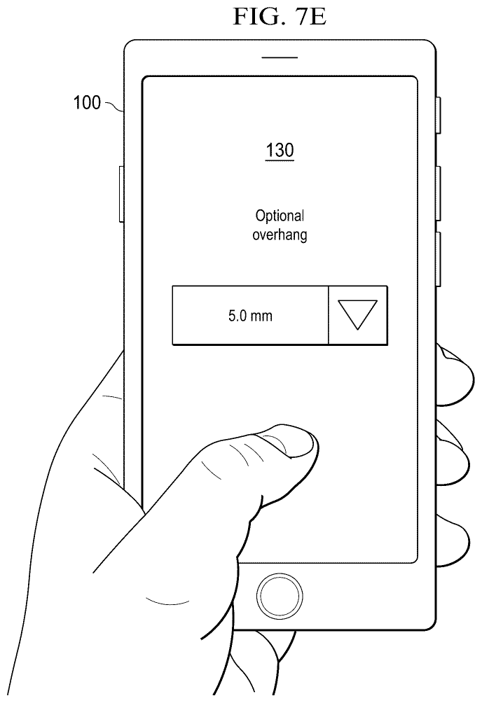

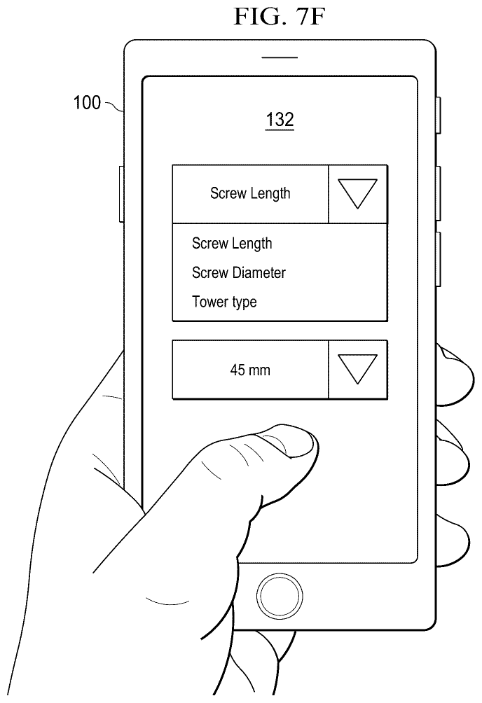

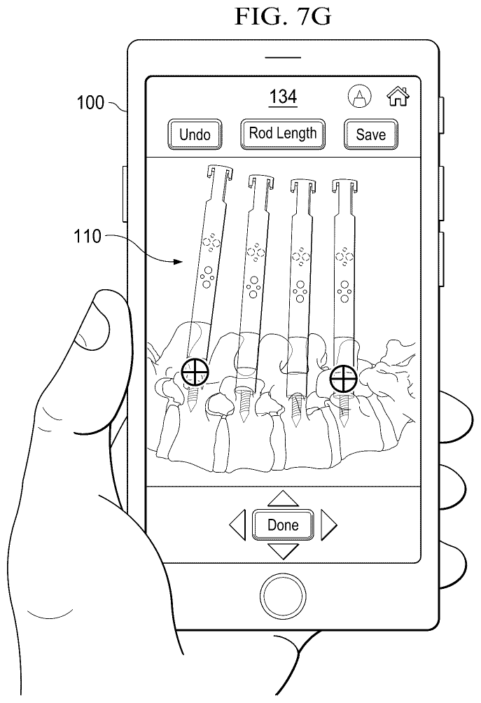

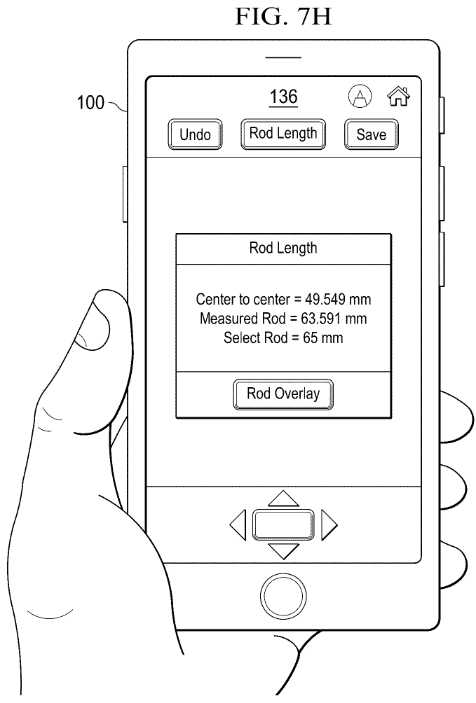

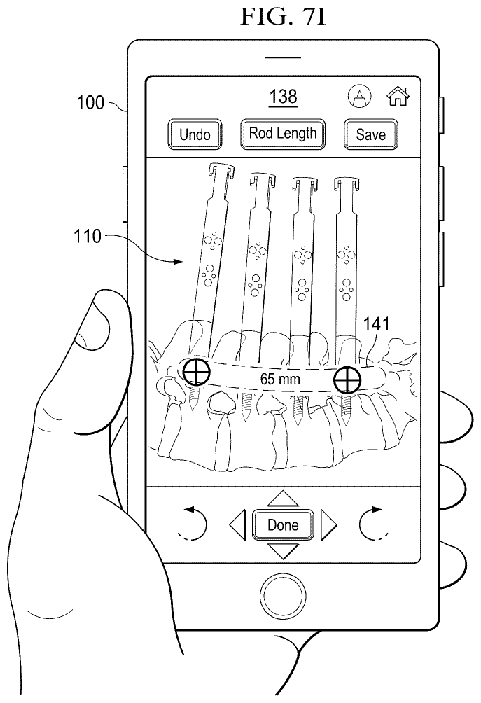

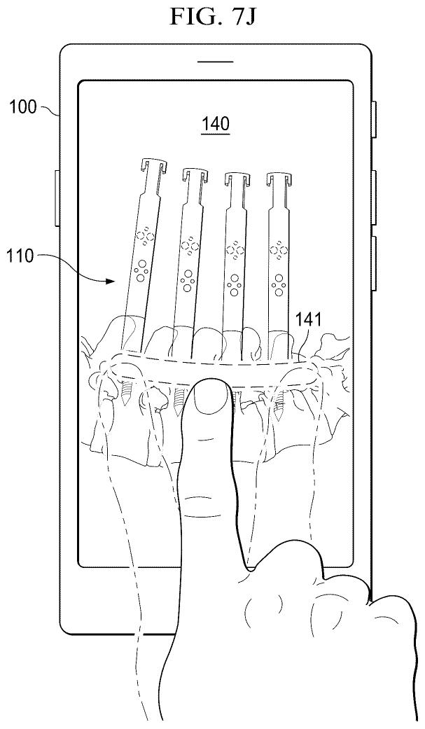

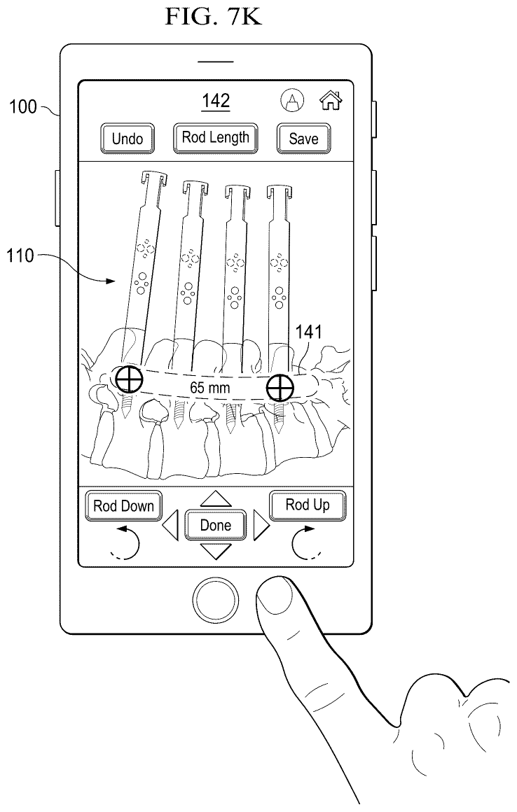

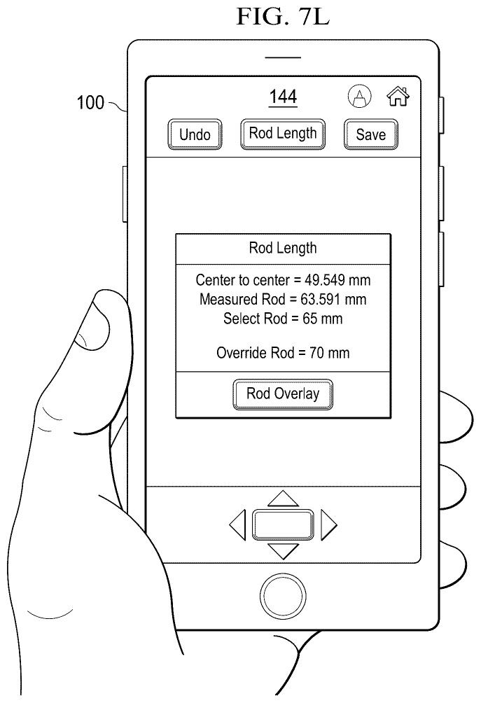

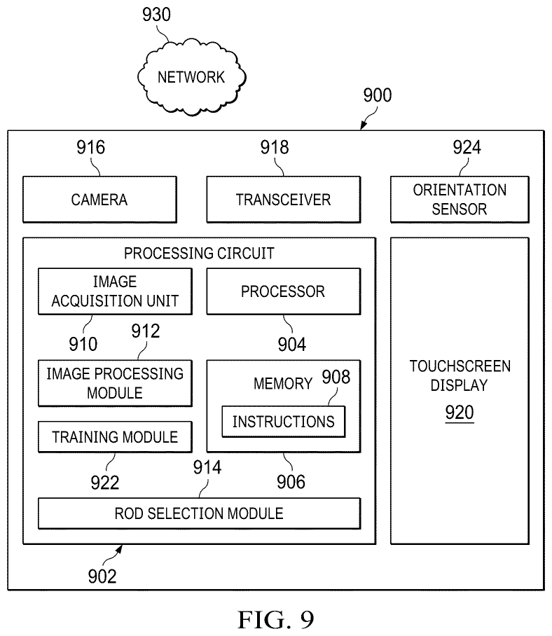

FIG. 6 is a flow diagram illustrating a computer-implemented method 600 for selecting a rod length based on one or more radiographic images of a patient's spine, according to aspects of the present disclosure. The method 600 may be performed using a mobile computing device, such as the mobile computing device 100 shown in FIG. 1 or the mobile computing device 900 shown in FIG. 9. FIGS. 7A-7L illustrate various steps of the method 600.

Referring to FIGS. 6, 7A, and 7B, at step 602, the mobile computing device 100 receives one or more radiographic images 110 of at least one fixation device and at least one radiographic marker. In the illustrated embodiment, the mobile computing device 100 includes a smartphone having a touchscreen display. The radiographic markers are incorporated into the tower receiver bodies. For example, the radiographic markers may include holes and/or slots drilled through the tower bodies to reduce the radiopacity of the tower body in the marker area. In other embodiments, the radiographic marker may include an external radiopaque marker attached to the skin of the patient (e.g., as in FIG. 1). In still other embodiments, the tower bodies themselves, or a feature or shape of the tower bodies, may be considered a radiographic marker. For example, the mobile computing device 100 may be configured to detect one or more edges of the tower bodies to determine a height, width, and/or any other relevant dimension of the tower body.

In some aspects, step 602 includes obtaining the image using a camera of the mobile computing device 100. In this regard, FIG. 7A shows the mobile computing device 100 having a user interface 122 for selecting a view before obtaining a radiographic image. The user interface 122 includes multiple buttons corresponding to different views of the patient, different implant types (e.g., Implant A, B, C, etc.), and doctor information. The views may include lateral and A/P. In other embodiments, the user interface 122 may include, instead of or in addition to those shown in FIG. 7A. a P/A button, and oblique view button, and/or any other suitable view button. In the illustrated embodiment, the user interface 122 further includes an optional view previous report button. In this regard, the user may choose from previously selected data. For example, the user may cause the mobile computing device 600 to perform the method multiple times to estimate or select a rod length multiple times. The user may wish to compare a previously determined rod length from a previous image to a later-determined rod length.

The mobile computing device 100 then receives the radiographic image 110 of the spine and one or more pedicle screw assemblies. In the illustrated embodiment, the view is a lateral view. In other embodiments, the view may be an A/P view, a P/A view, or any other suitable view. Referring to FIG. 7B, a user interface 124 includes a live view or “viewfinder” and a Capture button for capturing the image 110. In this regard, obtaining the image 110 may include directing the camera to a display in the surgical environment (e.g., a boom display), where the display is showing a radiographic image of the spine and pedicle screw assemblies. Thus, the image obtained in step 602 may be a reproduction of the image shown on a medical display in the operating environment. The mobile computing device may take an image of at least a portion of the display that shows the pedicle screw assemblies and spine. In some aspects, the user interface 124 further includes an indicator of the tilt of the mobile computing device to assist the user in obtaining the image along a plane that is parallel to the display.

In some aspects, step 602 includes selecting views and capturing images multiple times. For example, each captured image may be associated with a different view. Accordingly, the user may repeat the actions illustrated in FIGS. 7A and 7B multiple times, in some embodiments. For example, step 602 may include obtaining or capturing a first radiographic image associated with a first view and a second radiographic image associated with a second view different from the first view. The different views of the first and second radiographic images may provide for a parallax determination to better determine the location and/or dimensions of the image features, such as the rod connection points discussed below.

In some embodiments, step 602 includes receiving radiographic images directly from a radiographic imaging system (e.g., x-ray imaging system). For example, the radiographic imaging system may include an interface (e.g., interface 30, FIG. 1), where the interface is configured to communicate one or more radiographic images to the mobile computing device 100. For example, the interface may include a wired data port (e.g., universal serial bus (USB), Apple® LIGHTNING, FIREWIRE, Ethernet, etc.). In another example, the interface may include a wireless interface, such as Bluetooth®, Wi-Fi™, UWB, NFC, LTE, and/or any other suitable wireless interface. In some aspects, the interface may include a network connection or internet connection. For example, the interface of the radiographic imaging system may be in communication with a network computer capable of sending the one or more radiographic images via email, SMS text, cloud storage link, and/or any other suitable communication interface method.

Referring to FIGS. 6, 7C, and 7D, in step 604, the mobile computing device 100 calibrates image dimensions and/or orientations based on the radiographic marker. In some aspects, step 604 includes the mobile computing device 100 identifying, in the one or more radiographic images, the one or more radiographic markers, and obtaining one or more measurements of the one or more radiographic markers. The measurements may include a diameter, width, height, eccentricity, and/or any other dimension of the one or more radiographic markers. In some aspects, the measurements may be relative measurements, and may be in unites of pixels, or in a fraction or percentage of the field of view of the image. In some aspects, step 604 may include determining a conversion factor or calibration factor based on the one or more measurements of the radiographic markers. For example, the mobile computing device 100 may retrieve, from a memory, a known size of the one or more radiographic markers (e.g., in millimeters, inches, etc.), and determining the conversion factor or calibration factor by comparing the relative measurements (e.g., in pixels) to the known measurements).

Referring to FIG. 7C, calibrating the image dimensions may include selecting a “calibrate” option on a user interface 126. In some embodiments, once the “calibrate” button has been selected on the user interface 126, the user may use the directional arrows to overlay a shape, such as a bullseye, circle, rectangle, ellipse, and/or any other suitable shape, onto an object of a known size in the image 110. For example, in the embodiment illustrated in FIG. 7D, a user interface 128 includes the image 110, the directional arrows, and a “done” button. The user interface 128 also includes an “undo” button, “rod length” calculation button, and “save” button. The user uses the directional arrows to place the bullseye target over the screw body associated with an implant type. The screw body may be associated with one or more known dimensions, such as a diameter, width, height, length, and/or any other suitable dimension. The known dimensions may be stored or otherwise indicated on the device 100. The known dimensions may be based on a selected implant or implant type, such as an implant type selected using the user interface 122 in FIG. 7A. In other embodiments, the known dimension may be a screw shank length, a screw body height, and/or any other suitable dimension. In other embodiments, the object may not be an implant. For example, the object may be a radiopaque marker as explained above. The known dimension may be a diameter, width, and/or any other suitable dimension of the marker.

In some aspects, calibrating the image 110 also includes resizing the overlaid shape (e.g., bullseye) to match the known dimension in one or more respects. For example, referring to FIG. 7D, the user may use the touch display of the device 100 to resize at least one of the image 110 or the bullseye marker so that the diameter of the bullseye marker matches, or substantially matches, the width of the receiver body at the base. In other embodiments, the user may use the touch display to resize graphical marker to match the size and/or shape of a radiopaque marker, the screw shank height, screw body height, and/or any other suitable dimension. When the user is satisfied with the size and position of the marker, the user may select the “done” button to complete the calibration. In other embodiments, the user may place multiple markers on multiple objects in the image to perform the calibration.

Referring to FIGS. 6 and 7E and 7F, in step 606, the mobile computing device 100 receives one or more user inputs indicating one or more rod selection parameters. The one or more rod selection parameters may include, for example, optional overhang, screw length, screw diameter, tower type, amount of curvature or lordosis, and/or any other suitable parameter associated with selecting the size of the rod. Accordingly, step 606 may include using one or more of the user interfaces 132 and 134 to select and input one or more rod selection parameters. For example, FIG. 7E illustrates a user interface 130 by which a rod selection parameter, optional overhang, can be selected. In some aspects, the optional overhang interface object may include a drop down menu, a number pad, text input field, or any other suitable interface object for selecting the optional overhang. The optional overhang may be indicated in millimeters, inches, percent of rod length, and/or any other suitable method of measurement. FIG. 7F illustrates a user interface 132 by which one or more pedicle screw parameters can be selected. For example, a screw length, a screw diameter, and/or a tower type may be input using the user interface 132. In some aspects, the screw length and/or screw diameter may be selected by the user by selecting one of a plurality of preset of preconfigured screw length and/or screw diameter options. For example, the user may select a screw length of 25 mm, 30 mm, 35 mm, 40 mm, 45 mm, 50 mm, 60 mm, and/or any other suitable length. The screw diameter may be similarly selected from a set of screw diameters including 3 mm, 4 mm, 5 mm, 6 mm, 7 mm, and/or any other suitable screw diameter, both greater or smaller. The “tower type” may be selected from a set of stored tower types. The tower types may be indicated and selected based on a tower feature, tower size, product number, or any other suitable identifier.

Referring to FIGS. 6 and 7G, at step 608, the user uses the mobile computing device 100 to identify, based on the one or more radiographic images, the outer rod connection points. FIG. 7G shows the mobile computing device 100 displaying a user interface 134, where the user interface 134 shows the image 110 and allows for connection points to be placed on the image at locations corresponding to a desired rod connection point. For example, during the surgical procedure and before all of the pedicle screw assemblies have been coupled to the patient's spine, user may indicate on a touch screen of the mobile computing device 100 where are the rod connection points. In another embodiment, the mobile computing device 100 may determine, by analyzing the image 110, one or more rod connection points corresponding to the placed pedicle screw assemblies. The user may add a connection point or “entry point” by moving a cursor, crosshairs, or other graphical indicator onto the screen at the desired location. The mobile computing device 100 may then store or log the selected entry point in a memory device for subsequent use in the rod length calculation. In some aspects, the mobile computing device 100 may be configured to preliminarily select a rod connection point or entry point, and the user interface 134 may allow the user to reposition or adjust one or more of the rod connection points. The mobile computing device 100 may then store the adjusted rod connection points.

As explained above, one or more rod connection points or entry points may be identified by the mobile computing device 100 by identifying one or more components of a pedicle screw assembly, and locating the connection point based on a reference feature of the pedicle screw assembly. In other embodiments, a rod connection point may be input by a user to the mobile computing device 100 using a user interface (e.g., user interface 130 in FIG. 7E). Accordingly, the mobile computing device 100 may be configured to combine both computer-identified connection points and user-identified connection points to determine the span of the rod. In other embodiments, both connection points, or all connection points, may be selected or identified by the user via the user interface 134. In some embodiments, the interface 134 may also include a “change view” button if a different image having a different view would be better for identifying the connection points.

Referring to FIGS. 6 and 7H, at step 610, the mobile computing device 100 determines or selects the rod length based on the outer rod connection points and one or more rod selection parameters. In some embodiments, step 610 may include the mobile computing device 100 calculating, based on the calibration factor and the image data, the distance between the outer connection points. For example, calculating the rod length may include multiplying a relative distance (e.g., in pixels) between the outermost rod connection points by the calibration factor. Step 610 may further include the mobile computing device 100 adding the selected optional overhang to the calculated distance. In some aspects, step 610 may be performed by the mobile computing device 100 based on a selected screw length, screw diameter, tower type, lordotic curvature, and/or any other suitable parameter. For example, in some embodiments, the calibration factor may be calculated by measuring a relative length of a bone screw in the image (e.g., in pixels), and comparing the relative length to the screw length indicated by the user (e.g., 45 mm). In some aspects, the calibration factor may be determined based on a combination of measurements of the radiographic marker and one or more features of the pedicle screw assemblies, such as the screw length, tower length, screw diameter, tower width, and/or any other suitable value. As shown in FIG. 7H, the rod length selected at step 610 may be output by the mobile computing device 100 to a user interface 136. In some aspects, the mobile computing device 100 may output the selected rod length, as well as one or more calculation results on which the selected rod length is based. For example, the user interface 136 of FIG. 7H shows the selected rod length, as well as the distance between the centers of the selected points, and the “measured rod” dimension. In some aspects, the mobile computing device 100 may be configured to round up from the measured rod calculation to the closest rod in increments. The rod lengths may be provided in increments of 1 mm, 5 mm, 10 mm, and/or any other suitable increment.

Referring to FIGS. 6 and 7I at step 612, the mobile computing device 100 overlays a graphical indicator of a rod having the selected rod length onto the image 110 of the spine and the one or more pedicle screw assemblies. FIG. 7I shows a user interface 138 that includes the image 110 and the overlayed graphical indicator 141 positioned to span the outermost rod connection points. In some embodiments, step 612 includes generating the graphical indicator 140 of the rod based on the rod length selected in step 610. Step 612 may further include adjusting a size of the graphical indicator 141 to scale the graphical indicator with the image 110. For example, the size of the graphical indicator 141 may be adjusted based on the calibration factor determined at step 604. In some aspects, the mobile computing device 100 outputs the graphical indicator 141 on the user interface 138 at any suitable position (e.g., near the center), such that the user can orient and position the graphical indicator 141 to span the outermost rod connection points. This may represent the desired rod position after implanting and securing to the pedicle screw assemblies. In other embodiments, the mobile computing device 100 may be configured to automatically position and orient the graphical indicator 141 based on the graphical indicators. For example, the computing device may be configured to determine a midpoint between the outermost rod connection points, and a line extending between the outermost rod connection points. The mobile computing device 100 may be configured to overlay the graphical indicator 141 such that a longitudinal axis of the graphical indicator aligns with the line, and such that a center point of the graphical indicator aligns with the midpoint between the outermost rod connection points. In another embodiment, the mobile computing device 100 may be configured to overlay the graphical indicator 141 such that a first rod connection location on the rod aligns with a first outermost rod connection point, and such that a second rod connection location on the rod aligns with a second outermost rod connection point on the image 110. In some aspects, the user may position or modify the position of the graphical indicator 141 using the touch screen interface. For example, the user may rotate the graphical indicator using a two-finger gesture, and may move the graphical indicator using a one-finger gesture. In the illustrated embodiment, the user interface 138 may further include or indicate the selected rod length. For example, the selected rod length may be included on the graphical indicator 141. The user interface 138 includes directional arrows for shifting, translating, and rotating the graphical indicator 141 to an acceptable position. Once the user is satisfied with the positioning, the user may select the “done” button to advance to the next step.

Referring to FIGS. 6, 7J, and 7K, at step 614, the mobile computing device 100 receives inputs indicating a rod curvature. In some aspects, the inputs may indicate additional, or less, curvature. For example, the user may determine, based on the overlayed graphical indicator 141 and the image 110, that the curvature of the graphical indicator 141 does not match the profile of the rod connection points at each of the pedicle screw assemblies in the image 110. Thus, the user may user a multi-touch gesture to adjust the curvature of the graphical indicator 141. For example, FIG. JH shows a user interface 140 whereby the user is placing two fingers at or near the ends of the graphical indicator 141, and a single finger is positioned near the center of the graphical indicator 141 to flex the graphical indicator 141 up or down, thereby adjusting the curvature. Once the curvature has been set, the user may re-align the graphical indicator 141 with the connection points to verify that the graphical indicator 141 adequately overlays or overlaps the connection points. In another embodiment, the user may adjust the curvature using a dial indicator and/or “+” and “−” buttons to increase or decrease the amount of curvature of the graphical indicator 141.

As shown in FIG. 7K, a user interface 142 is shown in which the rod position, orientation, and/or curvature has been adjusted according to steps 612 and 614. The user interface 142 includes “rod down” and “rod up” buttons allowing for the user to replace the graphical indicator 141 with a graphical indicator of a rod of smaller or larger size. For example, based on the rod curvature and/or repositioning of steps 612 and/or 614, the user may desire to view how a larger rod would align with the implants in the image 110. Once the user is satisfied with the curvature, positioning, and length of the rod as illustrated in the user interface 142, the user may select the “done” button to confirm the changes. If the user did not change the rod size from what was initially determined, selecting the “done” button may confirm the initial rod length selection.

In another embodiment, the mobile computing device 100 may be configured to automatically adjust the curvature of the graphical indicator 141 by identifying a rod connection point for each pedicle screw assembly, or for each vertebral body to which the pedicle screw assemblies will be attached. For example, the mobile computing device 100 may be configured to adjust a fit function associated with the graphical indicator 141 until the graphical indicator 141 overlaps all the connection points.

Referring to FIGS. 6 and 7L, at step 616, the mobile computing device 100 determines whether to reselect a rod length based on the user inputs and adjusted curvature determined at step 614. In this regard, in some aspects, if the rod curvature is increased, the linear length or span of the rod may decrease. In some instances, the decreased linear length may cause the rod length to fail to meet the selected overhang requirements, for example. In another example, if no overhang is desired, the decreased linear length may cause the rod length to not span the full length between the outermost rod connection points. Accordingly, the mobile computing device 100 may be configured to increase the rod length to satisfy the overhang and any other rod selection parameters, and output the increased rod length. The user interface 144 may show the override, or newly-selected, rod length based on steps 612, 614, and/or 616. In some aspects, the method 600 may not include step 616. In other aspects, step 616 may include the user selecting the rod length such that the mobile computing device 100 may not determine the rod length.

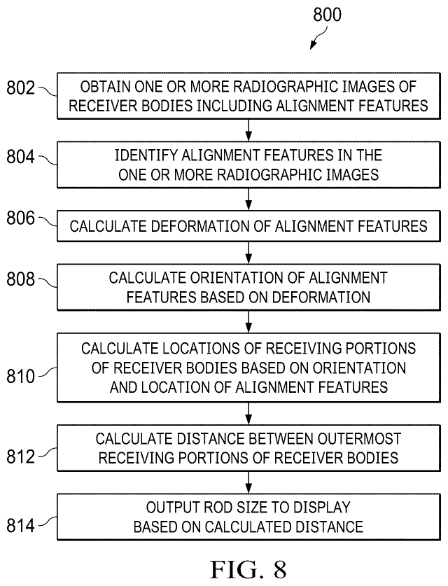

FIG. 8 illustrates a method 800 for determining or selecting a rod length or rod size. according to another embodiment of the present disclosure. In some aspects, the method 800 may involve similar steps, actions, and/or devices as the method 600 described with respect to FIG. 6. For example, the method 800 includes obtaining one or more radiographic images of one or more pedicle screw receives bodies, calculating connection points or receiving portions of the receiver bodies, and outputting the rod size to a display based on a calculated distance. In the method 800, the pedicle screw receiver bodies or towers may include integrated alignment features that can facilitate the detection or calculation of the rod connection points, which may be referred to as the receiving portion of the receiver bodies. The alignment features may include, for example, patterns or indicia drilled or cut into the receiver bodies as illustrated in FIGS. 5A-5D. In some embodiments, the alignment features may include one or more edges or borders of the receiver bodies. It will be understood that one or more steps of the method 800 may be performed using the mobile computing device 100 described above, and/or the computing device 900 described below with respect to FIG. 9. For example, the actions of method 800 may be performed by a smartphone, a tablet, personal data assistant (PDA), and/or any other suitable computing device.

At step 802, a mobile computing device obtains one or more radiographic images of a patient's spine including one or more receiver bodies. At least one of the receiver bodies may include one or more alignment features. The alignment features may be used to calibrate or scale the image, and/or to determine the location of the receiving portions of the receiver bodies. The receiving portions of the receiver bodies may be a saddle or channel at which the rod can be seated and tightened down to fix the rod to the receiver bodies.

At step 804, the mobile computing device identifies one or more alignment features in the one or more radiographic images. In some aspects, the mobile computing device uses image processing and analysis techniques to search the image for shapes and objects matching a type of profile. In some aspects, step 804 includes edge or boundary detection, segmentation, and/or any other suitable image analysis technique to identify the alignment features. In some aspects, step 804 includes determining a reference dimension or calibration factor based on the size of the alignment features in the image. For example, the mobile computing device may store a known absolute size (e.g., width, height, diameter) of the one or more alignment features in a memory, and may compare the known absolute size to a determined relative size (e.g., in pixels) to determine the calibration factor.

At step 806, the mobile computing device calculates, based on the one or more radiographic images, a distortion of the one or more alignment features. For example, if the alignment feature is a circle, the mobile computing device may be configured to determine an eccentricity of a detected ellipse corresponding to the detected alignment feature. In another example, if the alignment feature is a square, the mobile computing device may determine the distortion of the alignment feature based on a comparison of the width, height, and tilting angle of the alignment feature. At step 808, the mobile computing device determines an orientation of the alignment features based on the distortion determined at step 806. a tilt of the alignment feature in one or more dimensions (e.g., X, Y, Z). In some aspects, the mobile computing device may determine a relative spacing between components of the alignment features, and compare the relative spacing between the alignment features to the relative sizes of the alignment features. Because the alignment features are incorporated into the structure of the receiver bodies, the tilt and orientation of the receiver bodies may be inferred by the tilt and orientation of the alignment features.

At step 810, the mobile computing device calculates the locations of one or more receiving portions of the receiver bodies based on the orientation and location of the alignment features as determined in steps 804-808. In some aspects, step 810 may be performed based on geometrical information of the alignment features, the receiver bodies, and/or the bone screws attached to the receiver bodies. For example, the mobile computing device may retrieve, from a memory or a database, a relative spacing between the alignment feature and the receiving portion. Based on the known relative spacing, the mobile computing device may calculate or estimate the location of the receiving portion.

At step 812, the mobile computing device calculates a distance between the receiving portions of the outermost receiver bodies. In some aspects, step 812 is performed based on a calibration factor determined at step 804.

At step 814, the mobile computing device calculates or selects a rod size based on the distance calculated at step 812. In some aspects, the rod size may be calculated further based on one or more rod selection parameters as described above. For example, the mobile computing device may determine the rod size based on the distance calculated at step 812 and a selected overhang.

FIG. 8 illustrates a method 800 for determining or selecting a rod length or rod size, according to another embodiment of the present disclosure. In some aspects, the method 800 may involve similar steps, actions, and/or devices as the method 600 described with respect to FIG. 6. For example, the method 800 includes obtaining one or more radiographic images of one or more pedicle screw receives bodies, calculating connection points or receiving portions of the receiver bodies, and outputting the rod size to a display based on a calculated distance. In the method 800, the pedicle screw receiver bodies or towers may include integrated alignment features that can facilitate the detection or calculation of the rod connection points, which may be referred to as the receiving portion of the receiver bodies. The alignment features may include, for example, patterns drilled or cut into the receiver bodies as illustrated in FIGS. 5A-5D. In some embodiments, the alignment features may include one or more edges or borders of the receiver bodies. It will be understood that one or more steps of the method 800 may be performed using the mobile computing device 100 described above, and/or the computing device 900 described below with respect to FIG. 9. For example, the actions of method 800 may be performed by a smartphone, a tablet, personal data assistant (PDA), and/or any other suitable computing device.

Although described as being performed by a mobile computing device, it will be understood that one or more aspects of the methods 600 and/or 800 may be performed by a remote computing device other than the mobile computing device. For example, in some aspects, the mobile computing device 100 is configured to transmit image data and/or rod selection parameters input into the mobile computing device, via a network, to a remote server. The remote server may be configured to perform one or more actions of the methods 600, 800 to determine or select a rod length, adjust a rod curvature, generate a graphical indicator of the rod, and/or adjust the rod length based on the curvature. Accordingly, the mobile computing device 100 may operate in concert with one or more remote computing devices to determine the rod length. In some embodiments, the remote computing device may determine the rod length based on historical measurements and training data obtained and organized using artificial intelligence or machine learning techniques. For example, the remote computing device may be configured to receive the image and rod selection parameters from the mobile computing device, input the image data and rod selection parameters into a machine learning algorithm bolstered by machine learning data, and transmit, to the mobile computing device, an indication of the determined rod length.

FIG. 9 illustrates a mobile computing device 900 according to an embodiment of the present disclosure. The mobile computing device 900 may be configured to perform various actions and/or methods for determining a rod length. For example, the mobile computing device 900 may be configured to perform one or more aspects of methods 600 and/or 800. The mobile computing device 900 may be the mobile computing device 90 described above, for instance. In some aspects, the mobile computing device 900 may include a smartphone, such as an iPhone® or Android® phone. In another aspect, the mobile computing device 900 may be a tablet, a laptop, a PDA, and/or any other suitable type of computing device.

The mobile computing device 900 includes a processing unit 902, a camera 916, a transceiver 918, and a touchscreen display 920. The touchscreen display 920 may be configured to output a user interface, such as one or more of the interfaces shown in FIGS. 7A-7L. The touchscreen display 920 may be further configured to receive one or more touchscreen inputs indicating selections by a user. For example, the touchscreen display may be configured to receive a touch input indicating a selection of a rod selection parameter, a location of a rod connection point, an adjustment to rod curvature, a positioning of a graphical indicator of a rod, capturing an image, associating an image with a view (e.g., A/P, lateral), and/or any other suitable input. The touchscreen display 920 may be in communication with the processing circuit 902, which may include a graphical processing unit (GPU) for outputting the user interface, and a one or more data interface components for receiving and processing user inputs.

The camera 916 may include a digital camera, such as a charge-coupled device (CCD) camera. The camera 916 may include an imaging sensor and one or more optical components, such as lenses, mirrors, and/or filters. The camera 916 may be suitable for obtaining one or more images of a medical display, for example the camera 916 may be in communication with the memory 906 of the processing circuit 902 for storing the one or more images. The transceiver 918 may include a wireless transceiver for communicating according to one or more radio access technologies (RATs) or wireless protocols, including Wi-Fi™, Bluetooth®, near field communication (NFC), ultra-wideband (UWB), long-term evolution (LTE), 5G New Radio (NR), and/or any other suitable type of wireless communication. The transceiver 918 may be configured for communicating with a network 930 for communicating data associated with the rod length selection methods. For example, the transceiver 918 may be configured to receive configurations and/or parameters for selecting a rod length. Further, as explained above, in some aspects, the mobile computing device 900 may be configured to operate in concert with a remote computing device of the network 930 to indicate the rod length to the user via the mobile computing device 900.

The processing circuit 902 includes a processor 904 and a memory 906 storing computer program instructions 908 executable by the processor 904. For example, the processor 904 may include a central processing unit (CPU), and the instructions 908 may include computer program code for performing one or more of the actions of methods 600 and/or 800. The processing circuit 902 also includes a plurality of modules and units, including an image acquisition unit 910, an image processing module 912, a rod selection module 914, and a training module 922. In some aspects, one or more of the units and modules of the processing circuit 902 may include physical hardware components (e.g., application specific integrated circuits (ASICs), field-programmable gate arrays (FPGAs), processors, etc.). In other aspects, one or more of the units and modules of the processing circuit 902 may include virtual modules facilitated by the instructions 908 of the memory 906, which modules are executable by the processor 904. In another aspect, the modules and units of the processing circuit 902 may include a combination of physical and virtual modules.

The image acquisition unit 910 may include hardware and/or software for receiving images representative of a patient's spine or other anatomies. In some aspects, the image acquisition unit 910 is configured to control acquisition of images using the camera 916. In another aspect, the image acquisition unit 910 is configured to receive images from a remote device (e.g., from a medical imaging system via the interface 30 in FIG. 1), and to provide the image data to one or more of the other modules of the processing circuit 902.

The image processing module 912 may include hardware and/or software for performing one or more image processing steps to identify features in the images received from the image acquisition unit 910. For example, the image processing module 912 may be configured to recognize and measure pedicle screw assemblies, spinal vertebrae, calibration markers, alignment features, and/or any other suitable image feature. For example, the image processing module 912 may be configured to perform one or more actions of steps 604, 608, 610, and/or 616 of the method 600. In another example, the image processing module 912 may be configured to perform one or more actions of steps 804, 806, 808, 810, 812, and/or 814 of the method 800. The image processing module 912 may be configured to use various techniques to perform these actions, including boundary detection, segmentation, object recognition, pattern recognition, feature extraction, filtering, point feature matching, and/or any other suitable technique.

The rod selection module 914 may include hardware and/or software for determining or selecting a rod length based on data extracted by the image processing module 912, and by user inputs from the touchscreen display 920. The rod selection module 914 may be configured to determine the rod length according to an algorithm or formula stored in the instructions 908. For example, the rod selection module 914 may be configured to select the rod length based on a sum of the computer-determined distance between two rod connection points, and two-times a selected overhang length. The overhang length may be selected by the user, or may be preconfigured in the instructions 908. In some aspects, the rod selection module 914 may be configured to output the selected rod length to the touchscreen display 920.

The training module 922 may include hardware and/or software for improving rod length selection using machine learning data. For example, the training module 922 may be configured to store, update, and output a model based on training data. The training data may be received from the network 930, and may be associated with historical rod selection procedures obtained over time. Further, the training module 922 may be configured to prepare training data based on the rod selection performed by the modules of the processing circuit, and to transmit the training data to the network 930 to update a machine learning model. In some aspects, if the user determines that the rod selection module 914 did not output a correct rod length, or that the algorithm for rod length selection could benefit from a correction, the training module 922 may be configured to receive inputs from the user associated with the correction, and update the machine learning model based on the corrections.

The mobile computing device 900 may further include an orientation sensor 924. In some aspects, the orientation sensor 924 includes an accelerometer. In other aspects, the orientation sensor 924 includes a gyroscope. In other aspects, the orientation sensor 924 includes both an accelerometer and a gyroscope. The orientation senso 924 may be configured to obtain orientation data, and provide the orientation data to the processing circuit 902. For example, the orientation data may indicate or represent an angular orientation of the mobile computing device 900 relative to a horizontal plane, or to a vertical plane. In some aspects, the processing circuit 902 of the mobile computing device 900 may be configured to perform one or more aspects of the methods 600, 800 described above based on the orientation data. For example, if the camera 916 of the mobile computing device 900 is used to obtain a photo of a medical display showing the radiographic image, the processing circuit may be configured to take into account the orientation of the mobile computing device when determining the calibration factor, the rod connection points, and/or any other suitable aspect of the rod length selection methods. In another aspect, the processing circuit 902 may be configured to control the camera 916 based on the input from the orientation sensor 924. For example, the processing circuit 902 may be configured to disable an image capture feature until the orientation data from the sensor 924 indicates that the mobile computing device is within a certain range of angles from vertical. This may assist the user in obtaining an image of the medical display, where the field of view is on a plane parallel with the plane of the medical display.

As further explained above, in some instances, a rod selection algorithm may be performed by a combination of the mobile computing device 900 and a remote computing device (e.g., a server) of the network 930. For example, the mobile computing device 900 may server as an interface for the user in the surgical environment for obtaining the images, inputting rod selection parameters, visualizing a representation of the selected rod length on the display, and receiving additional inputs indicating a correction to the curvature and/or length of the graphical indicator of the rod. Further, one or more of the image processing and rod selection calculation steps may be performed wholly or in part by the remote computing device of the network 930, and may be transmitted to the mobile computing device 900 to indicate the user.

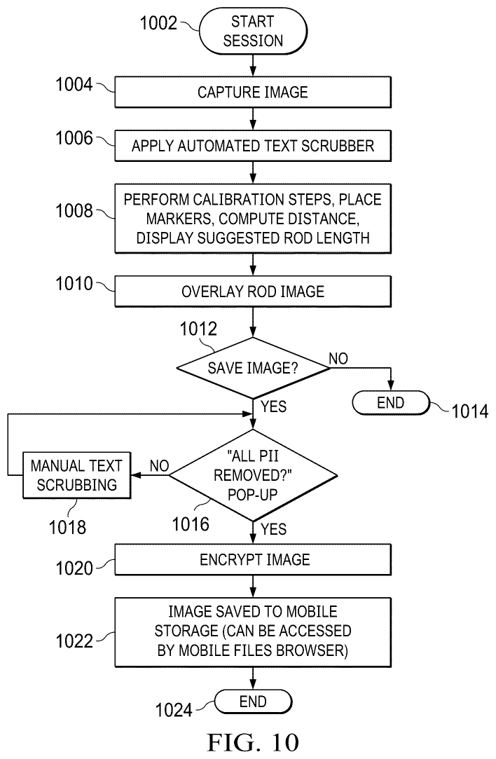

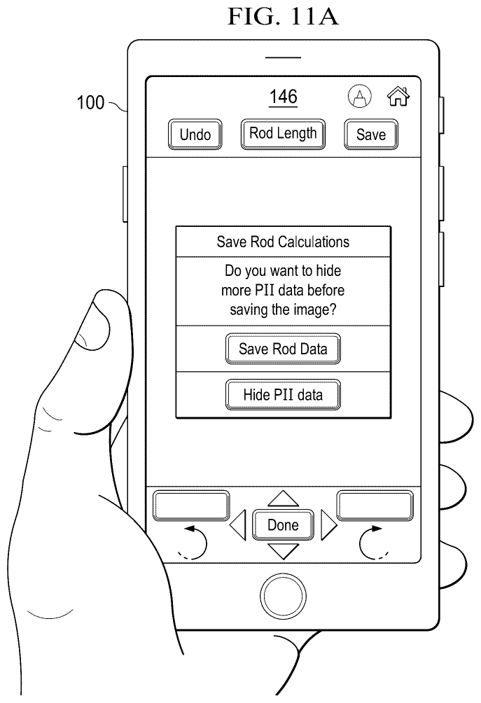

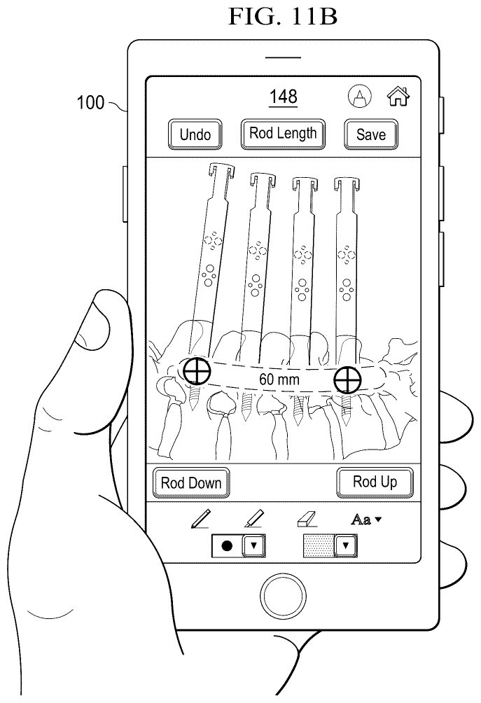

FIG. 10 is a flow diagram illustrating a method 1000 for selecting an interbody implant, according to aspects of the present disclosure. The method 1000 may include or involve aspects of the method 600 and/or the aspects shown in FIGS. 7A-7L. Additionally, the method 1000 may include or involve aspects of FIGS. 11A and 11B. In some aspects, the method 1000 may be performed using the computing device 100 and/or the computing device 900 in a spinal fixation or stabilization procedure. In some aspects, the method 1000 may be performed to determine, select, or predict a size of a spinal rod, a spinal bone screw, an intervertebral spacer, and/or any other suitable implantable device. Further, the method 1000 may include identifying and removing or redacting text from a captured image, and especially text providing patient-identifying information. In FIG. 10 and the description below, reference may be made specifically to spinal rods, and selecting a rod length. However, it will be understood that the method 1000 may be used to select any type of implant and any suitable size or dimension thereof.

At action 1002, a session is started. In some aspects, starting the session includes initiating a software application. For example, starting the session may include opening a smartphone application or “app.” In some aspects, the application may comprise an Apple IOS application, a Google Android application, a Microsoft Windows application, a web application, a Linux-based application, and/or any other suitable type of software application. In some aspects, starting or launching the application may include or involve initiating an image capture functionality. In another aspect, launching the application may involve authenticating the user using biometric authentication, password authentication, device id authentication, and/or any other suitable type of authentication. Starting the session may include or involve launching a user interface. The user interface may provide interactive interface objects, such as buttons, drop-down menus, text fields, and/or any other suitable type of interface object.

At action 1004, the method 1000 includes capturing an image. In some aspects, capturing the image includes inputting an image capture command on the mobile computing device. For example, the user may select a capture image button. In other aspects, the mobile computing device may capture the image automatically in response to detecting a suitable image in frame. In some aspects, the captured image may be an image of a computer monitor or television displaying human anatomy. For example, the mobile computing device may be configured to detect one or more shapes corresponding to a pedicle screw assembly, spinal anatomy, computer monitor, and/or any other suitable object. In response to detecting the object, the mobile computing device may capture the image. In some aspects, the image may be saved to random access memory. The image may comprise or represent spinal vertebrae, pedicle screw assemblies, intervertebral spacers, calibration markers, and/or any other suitable object in a field of view.

Referring to FIGS. 10 and 11A, at action 1006, the method 1000 comprises applying an automated text scrubber function. In this regard, the text scrubber may be configured to identify one or more text items in the captured image and to remove the captured text. In some aspects, applying the automated text scrubber comprises applying an optical character recognition (OCR) procedure on the image, and removing at least a portion of the identified characters. In some aspects, action 1006 includes removing only patient identifying information or text. For example, the mobile computing device may remove the patient's name, the physician, and/or any other information associated with the patient. In another aspect, action 1006 may involve removing, redacting, occluding, and/or otherwise scrubbing all text from the captured image.

FIG. 11A illustrates a user interface 146 which includes a selection menu for saving rod selection data. The user interface 146 includes an option to save rod data (e.g., selected rod length and associated calculations, etc.) and an option to hide patient identifying information (PII). In some aspects, selecting “Hide PII data” may initiate the automated text scrubber feature described above. In another aspect, selecting “Hide PII data” may initiate a manual text scrubbing feature whereby the user may manually delete the PII. In this regard, FIG. 11B illustrates a user interface 148 which provides a user with manual tools, such as a digital pencil/marker to write over and obscure text characters providing PII.

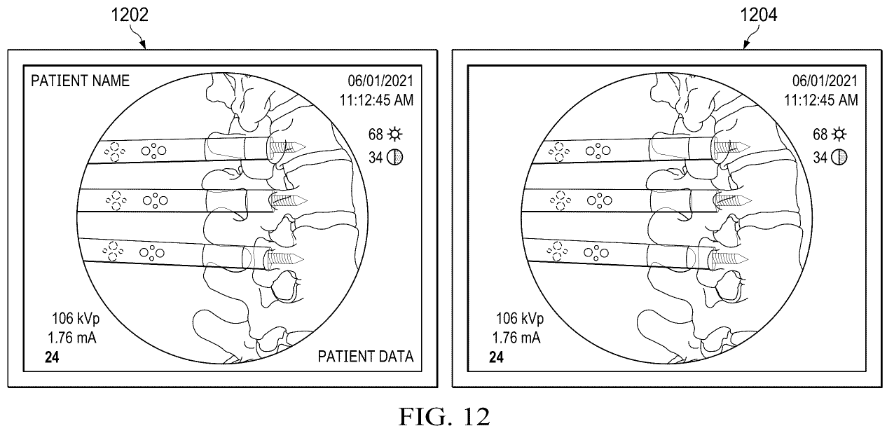

FIG. 12 illustrates an image 1202 before an automated text scrubber feature is applied, and the image 1204 after the automated text scrubber feature is applied. In the illustrated example, the automated text scrubber may eliminate or obscure only the text that is patient-identifying, such as “Patient Name” and “Patient Data.” Thus, the image 1204 removes the patient-identifying text or information and leaves other text and information on the screen, such as display settings, date, time, radiograph parameters, etc. As mentioned above, in other embodiments, the automated text scrubber may remove all identified text from the screen.

Referring again to FIG. 10, at action 1008, the method 1000 comprises performing one or more calibration steps, placing markers, computing a distance, and displaying a suggested rod length. In this regard, action 1008 may include or involve aspects of steps 604-610 of the method 600, and/or aspects of steps 804-814 described above.

At action 1010, the method 1000 includes overlaying an image of the rod on the screen. Aspects of action 1010 may include or involve aspects of step 612 of the method 600.

At action 1012, the method 1000 includes determining whether to save the captured image with the text removed. As explained above, in some aspects, capturing the image may include storing the captured image to a random access memory (RAM). Action 1012 may include determining whether to save the image to non-volatile storage on the mobile computing device, to a server, or both. In some aspects, the mobile computing device determines whether to save the image based on an input from the user. For example, the mobile computing device may output a screen prompting the user to select and option “yes” or “no,” where “yes” causes the mobile computing device to store the image. In some aspects, the determination of whether to save the image may be based on one or more questions and the corresponding response is provided by the user. For example, the method 1000 may include prompting the user to select whether the data should be stored for data analytics purposes.

At action 1014, in response to a determination not to save the image, the method 1000 is complete, in some aspects, action 1014 includes outputting, to a user display, an indication that the rod selection procedure is complete. In some aspects, action 1014 includes completing the session started at action 1002. At action 1016, in response to determining to save the image, the method 1000 proceeds to a second phase. In some aspects, the second phase involves a verification that all relevant text has been removed, redacted, or otherwise scrubbed from the image.

At action 1016, the method 1000 includes receiving an input from the user indicating whether all patient identifying information (PII) has been removed from the image. In some aspects, action 1016 includes displaying the image after the automated text scrubber has been applied according to step 1006. In some aspects, action 1016 includes outputting “yes/no” buttons on the screen via which the user can indicate whether the PII has been removed. In another aspect, action 1016 includes receiving a “continue” command from the user. For example, action 1016 may include providing a user interface whereby the user may manually remove any PII or other text, and if all PII has been removed, the user may select “continue.”

At action 1018, the method 1000 includes, in response to receiving an indication that not all PII has been removed, performing a manual text scrubbing operation. In some aspects, the manual text scrubbing operation comprises receiving inputs from the user identifying portions of the image associated with text that has not yet been removed. For example, action 1018 may include receiving user inputs identifying a bounding box or bounding area inside of which PII is contained. In another aspect, action 1018 includes a virtual eraser function or virtual marking function which blurs, obscures, blacks out, whites out, or otherwise scrubs remaining text in the image. After the user has manually scrubbed the text, a renewed prompt may appear on the screen allowing the user to indicate whether all PII has been removed from the image. The user may again select “yes” or “no” to indicate whether the PII has been removed. If the user indicates “no”, action 1018 is repeated. If the user indicates “yes,” or “continue,” the application may proceed to step 1020.

At action 1020, the method 1000 includes, in response to receiving an indication that the PII has been removed, encrypting the image.

At action 1022, the method 1000 includes saving or storing the encrypted image. In some aspects, storing the image may include storing the image to the mobile computing device. In another aspect, storing the image may include storing the image to a server. For example, storing the image may include transmitting the image to a remote server or data center. And another aspect, storing the image may include transmitting the image to a local server. In some aspects, the encryption of the image may allow the image to be accessed or viewed only on the same device which performed the steps of the method 1000. In another aspect, the encryption may include a password protection feature such that accessing the image or its associated data requires a password to be input.

At action 1024, the method 1000 is complete, and the session is terminated. In some aspects, action 1024 may include closing the software application. In some aspects, action 1024 may include clearing the mobile computing devices cache, erasing any images or data from any volatile or nonvolatile memory, and/or transmitting a user log to a server.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

What is claimed is:

1. A method of determining a size of a connecting rod performed by a mobile computing device, the method comprising:
obtaining, by an image acquisition unit of the mobile computing device, at least one radiographic image, wherein the at least one radiographic image is representative of two or more medical devices inserted into a body of a patient;
determining, by a processor of the mobile computing device based on at least one of the two or more medical devices in the at least one radiographic image, a calibration factor of the at least one radiographic image;
identifying, for the two or more medical devices, a first rod connection point and a second rod connection point;
determining, by the processor of the mobile computing device based on the first rod connection point, the second rod connection point, and the calibration factor, a length of the connecting rod associated with a distance between the first rod connection point and the second rod connection point; and
outputting, to a display, a visual representation of the length of the connecting rod.

2. The method of claim 1, wherein:
the at least one radiographic image is representative of a first medical device and a second medical device implanted into the patient,
the first medical device includes a first radiographic indicia and the second medical device includes a second radiographic indicia,
the method further includes:
determining, by the processor of the mobile computing device, a deformation of the first indicia; and
determining by the processor of the mobile computing device, a deformation of the second indicia;
the determining the first rod connection point is based on the first deformation; and
the determining the second rod connection point is further based on the second deformation.

3. The method of claim 2, further comprising:
determining a first relative length of the first indicia; and
determining a second relative length of the second indicia,
wherein the determining the first rod connection point is based on the first relative length of the first indicia, and
wherein the determining the second rod connection point is based on the second relative length of the second indicia.

4. The method of claim 2, wherein:
the first indicia comprises a first plurality of indicia features,
the determining the first deformation of the first indicia comprises determining:
a first relative distance between a first indicia feature and a second indicia feature of the first plurality of indicia features; and
a second relative distance between a third indicia feature and a fourth indicia feature of the first plurality of indicia features.

5. The method of claim 4, wherein the determining the first deformation of the first indicia comprises:
determining a first dimension of the first indicia feature;
determining a second dimension of the second indicia feature; and
comparing the first dimension to the second dimension.

6. The method of claim 1, further comprising receiving, from a user interface of the mobile computing device, a first input indicating the second rod connection point in the one or more radiographic images.

7. The method of claim 1, further comprising receiving, from a user interface of the mobile computing device, a second input indicating an overhang length of the connecting rod, wherein the determining the rod length is further based on the second input.

8. The method of claim 1, further comprising receiving, from a user interface of the mobile computing device, a third input indicating at least one of:
a type of the first medical device; or
a dimension of the first medical device,
wherein the determining the rod length is based on the third input.

9. The method of claim 1, wherein the determining the rod length comprises selecting, based on the first rod connection point and the second rod connection point, a stored rod length from a database including a plurality of stored rod lengths.

10. The method of claim 1, further comprising:
determining, by an orientation sensor of the mobile computing device, an orientation of the mobile computing device,
wherein the determining the calibration factor is based on the orientation of the mobile computing device.

11. The method of claim 1, further comprising:
outputting, to the display of the mobile computing device, a graphical indicator of a rod overlayed on a radiographic image of the one or more radiographic images, wherein the graphical indicator is scaled relative to the radiographic image based on the calibration factor.

12. The method of claim 11, further comprising:
receiving, from a user interface of the mobile computing device, a fourth input indicating an adjustment to a curvature of the connecting rod; and
updating the graphical indicator based on the adjustment to the curvature of the connecting rod.

13. The method of claim 12, further comprising:
determining, based on the fourth input, an updated rod length; and
outputting, to the display, a visual indication of the updated rod length.

14. The method of claim 1, further comprising:
receiving an indication to store the at least one radiographic image; and
removing, based on the indication, text from the at least one radiographic image.

15. The method of claim 14, wherein the removing the text comprises:
identifying, in the at least one radiographic image, patient-identifying information; and
removing text characters in the at least one radiographic image associated with the patient-identifying information.

16. The method of claim 14, further comprising:
encrypting, based on the indication, that at least one radiographic image; and
storing the encrypted at least one radiographic image to non-volatile memory.

17. A mobile computing device, comprising:
an image acquisition unit configured to obtain at least one radiographic image, wherein the at least one radiographic image is representative two or more medical devices inserted into a body of a patient;
a user interface; and
a processor in communication with the image acquisition unit and the user interface, the processor configured to:
determine, by a processor of the mobile computing device based on at least one of the two or more medical devices in the at least one radiographic image, a calibration factor of the at least one radiographic image;
identify, for the two or more medical devices, a first rod connection point and a second rod connection point;
determine, by the processor of the mobile computing device based on the first rod connection point, the second rod connection point, and the calibration factor, a length of the connecting rod associated with a distance between the first rod connection point and the second rod connection point; and
output, to a display, a visual representation of the length of the connecting rod.

18. The mobile computing device of claim 17, wherein the processor is further configured to receive, from the user interface of the mobile computing device, a second input indicating an overhang length of the connecting rod,
wherein the processor is configured to determine the rod length further based on the second input.

19. The mobile computing device of claim 17, wherein the processor is further configured to receive, from the user interface of the mobile computing device, a third input indicating at least one of:
a type of the first medical device; or
a dimension of the first medical device, 5
wherein the processor is configured to determine the rod length based on the third input.

20. The mobile computing device of claim 17, wherein the processor is further configured to:
output, to the user interface of the mobile computing device, a graphical indicator of a rod overlayed on a radiographic image of the one or more radiographic images, wherein the graphical indicator is scaled relative to the radiographic image based on the calibration factor.
